(19) United States
(12) Patent Application Publication
(10) Pub. No.: US 2015/0226724 A1
(43) Pub. Date: Aug. 13, 2015

(54) METHOD OF IDENTIFYING ENDOGENOUS FLUORESCENT BIOLOGICAL MARKERS FOR MONITORING CELLS

(71) Applicant: SOCPRA SCIENCES ET GENIE, S.E.C., Sherbrooke (CA)

(72) Inventors: Joël SIROIS, Sherbrooke (CA); Emmanuel BIZIER, Gatineau (CA); Guillaume CARDIN-BERNIER, Montreal (CA)

(21) Appl. No.: 14/601,287

(22) Filed: Jan. 21, 2015

Related U.S. Application Data
(63) Continuation of application No. 13/124,514, filed on Oct. 10, 2011.
(60) Provisional application No. 61/106,170, filed on Oct. 17, 2008.

(51) Int. Cl. G01N 33/483; G01N 21/64

(52) U.S. Cl. CPC G01N 33/4833; G01N 21/6486

(57) ABSTRACT

The present application related to methods for the identification of endogenous biological markers that generate information on the status of a cell. Because the techniques provided herein provide accurate results in a relatively short amount of time, the methods described herein can be used to identify numerous endogenous biological cell markers and monitor various cell culture conditions.

### CROSS-REFERENCE TO RELATED APPLICATIONS

This application claims priority on U.S. provisional application 61/106,170 filed on Oct. 17, 2008 and which is herewith incorporated in its entirety.

### BACKGROUND

In order to control and optimize a cell culture, different parameters (growth curves, consumption/depletion of nutrients, production/accumulation of by products (which are usually toxic), determination of physiological state) must be determined at various stages. However, some parameters of a cell culture are very difficult to quantify because there is only a limited number of methods for measuring them during laboratory experimentation or industrial production. Usually, samples of cell cultures are analyzed with conventional techniques (filtration, drying, cell counting, HPLC). These methods are generally lengthy and costly processes and cannot be performed in real time. Consequently, in industrial settings, cells are generally cultured using a pre-established recipe, based on a statistical indicator which can be indirectly linked to a specific cellular state. This strategy does not facilitate the real time optimization of cell cultures and results in important economic loss.

Some methods currently known in the art enable the determination of the biomass concentration in real time: probes measuring a NADH signal through the determination of its autofluorescence in cell culture, turbidity (such as the ASD19-N™ probe of Optek Danulat) and capacitance (such as the Biomass Monitor™ probe of Aber Instruments Ltd.). However, it is virtually impossible to determine in real time other important parameters of the cell culture such as cellular proliferation, physiological state, consumption of nutrients, production of a by-product, etc.

The fluorescence of a cell culture provides information about the status of a cell culture. However, the processing of this information to generate significant relationships with specific cell culture parameters is rather difficult. The main challenge in the identification of autofluorescent markers resides in the de-convolution of fluorescent signals obtained in real time. The signal obtained following an excitation of the cell suspension results in the sum of the individual signals emitted by multiple autofluorescent molecules contained in the cells and in the culture medium. Also, physical interactions between all the signals (ex.: interference and spectra overlapping) make the interpretation difficult.

Even though the use of endogenous fluorescence to determine the status of a cell culture has proved to be difficult, some research teams have published their efforts toward the understanding of this subject. Hisiger et Jolicoeur (2005, Biotechnological Progress, 21, 580-589) found eight unknown fluorescent compounds in Eschscholtzia californica culture, as well as signal surimposition of benzophenanthridic alkaloids and riboflavins. They also reported the relationship between NAD(P)H associated autofluorescence and cell activity in E. californica. They further reported the relationship between NAD(P)H associated autofluorescence and biomass in C. roseus. Finally, they noted the relationship between NAD(P)H associated and riboflavin associated autofluorescence and growth rate in C. roseus. Applicant would like to note that the tryptophane and tryptamine signals were inverted in their studies. Applicant would also like to point out that the same indicator (NAD(P)H) was correlated to two physiological variables which are linearly independent, which is problematic for the interpretation of results. Further, the conclusion reached by Hisiger and Jolicoeur was supported by only one reading which can lead to a erroneous interpretation of the real and reproducible correlations.

Hisiger and Jolicoeur (2005, Journal of Biotechnology, 117, 325-336) reported the relationship between NAD(P)H, riboflavin and tryptophane associated autofluorescence and biomass concentration in P. pastoris. They also suggested the relationship between riboflavin associated autofluorescence and biomass concentration in the NSO cell line. Surprisingly, even though riboflavin and tryptophane are not bio-synthesized by mammal cells, Hisiger and Jolicoeur pretend that it is <<possible>> to correlate the biomass concentration and the riboflavin associated autofluorescence.

Schalger et al. (1996, Advanced Space Research, 18, 113-124) developed algorythms to track microbial population evolution of Pseudomonas aeroginosa by auto-fluorescence. Schalger et al. reported estimation errors up to 42.9%.

The relationship between NAD(P)H and the growth of various cells has been disclosed by various groups. Asali et al. (1992, Biotechnology, 23, 83-94) reported the relationship between NAD(P)H associated fluorescence and biomass concentration in C. roseus. Farabegoli et al. (2003, Water Research, 37, 2732-2738) reported the relation between NAD(P)H associated autofluorescence and biomass concentration in active muds. The relationship between NAD(P)H associated autofluorescence and biomass concentration in C. botanica was also identified by Harrison et Chance (1970, Applied microbiology, 19, 446-450). Scheper et al. (1987, Annals New York Academy of Sciences, 506, 431-445) reported the relationship between NAD(P)H associated autofluorescence and cellular activity in various organisms. Siano et Mutharasan (1989, Biotechnology and Bioengineering, 34, 660-670) published the relationship between NAD(P)H associated autofluorescence and cellular activity in S. cerevisae.

Horvath et al. (1993, Biotechnology progress, 9, 666-670) reported the relationship between tryptophane associated autofluorescence and biomass concentration in S. cerevisae.

Li et Humphrey (1991, Biotechnology and Bioengineering, 37, 1043-1049) reported the relationship between NAD(P)H, riboflavin, tryptophane and pyridoxine associated autofluorescence and biomass concentration in C. utilis. The relationship between tryptophane associated autofluorescence and biomass concentration in S. cerevisae was also published by this group.

Also in S. cerevisae, Lindemann et al. (1998, Sensors and actuators B, 51, 273-277) reported the relationship between riboflavin associated autofluorescence and biomass concentration.

Palmer et al. (2003, Photochemistry and photobiology, 78, 5, 462-469) published the relationship between tryptophane associated autofluoescence and cellular concentration in human mammal cells. Their correlation was not used to monitor cell culture, but to discriminate between malignant and normal cell lines.

Some endogenous autofluorescent markers have been shown to be correlated with biomass concentration in some cell culture. However, information regarding to biomass concentration/accumulation provides only partial information about the cell culture. For example, it does not provide information about the metabolic behavior of the cells in culture (ex.: cell proliferation, nutriments consummation and use, metabolic activity, etc.).

In light of the above, it would be highly desirable to be provided with a method for identifying markers that accurately represent one or several parameters of a cell culture. The method should enable the identification of markers that are rapidly measured in order to provide real time or quasi real time information on the status of the cell culture. The method should also identify markers that are able to represent different culture parameters in order to provide rapidly very important information on the status of the cell culture.

### BRIEF SUMMARY

The results presented herewith results show that autofluorescent markers that can be measured during a cell culture are associated with the modulation of a cell culture parameter. The present application thus provides methods for identifying those endogenous fluorescent markers.

According to a first aspect, the present application provides a method of identifying an endogenous fluorescent marker associated with a parameter of a cell. The method broadly comprises first determining if an optical imprint of the endogenous fluorescent marker is associated with a fluorescent signal from the cell. The method then comprises providing a value for the parameter of the cell culture. The method also comprises correlating the optical imprint associated with the fluorescent signal from the cell with the value of the parameter of the cell in order to identify the endogenous fluorescent marker. In an embodiment, the endogenous fluorescent marker is associated with an endogenous biological marker of the cell. In another embodiment, the optical imprint of the endogenous biological marker is from a three-dimensional fluorescent spectra. In yet another embodiment, a fluorescent signal is measured from the cell. In still another embodiment, the fluorescent signal is from a fluorescent reading at a selected excitation wavelength and/or at a selected emission wavelength. In yet another embodiment, the fluorescent signal is obtained online, such as, for example, with a probe that can be optionally integrated in a cell culture vessel. In yet another embodiment, the optical imprint is obtained from a chemically purified form of the endogenous fluorescent marker. In still another embodiment, the endogenous biological marker is known to be produced or consumed by the cell. In yet another embodiment, the cell is a cultured cell. In still another embodiment, the fluorescent signal is from a culture of the cell or a derivative of such culture, such as, for example, an untreated sample of the culture, a filtered sample of the culture and a resuspended retentate of the filtered sample. In an embodiment, the correlation can be determined graphically, by a numerical technique (such as, for example, a covariance coefficient technique) and/or by a multivariable technique. In yet another embodiment, the parameter is at least one of biomass concentration, rate of biomass accumulation, cellular concentration, rate of cellular proliferation, accumulation of a by-product and consumption of a nutrient. In an embodiment, the endogenous fluorescent marker is not NAD(P)H.

According to another aspect, the present application relates to a method of estimating a parameter of a cell from a fluorescent signal of an endogenous biological marker. Broadly, the method first encompasses identifying the endogenous fluorescent marker with the method described above. Then the method comprises obtaining a fluorescent signal for the endogenous fluorescent marker. The method further comprises obtaining a value for the parameter of the cell. The method also comprises determining a mathematical relationship between the fluorescent signal for the endogenous fluorescent marker and the value for the parameter of the cell as well as estimating the parameter of the cell culture value based on the mathematical relationship. In an embodiment, the mathematical relationship is a linear regression and, in yet a further embodiment, the estimation is generated with the slope of the linear regression. In still another embodiment, the parameter is at least one of biomass concentration, rate of biomass accumulation, cellular concentration, rate of cellular proliferation, accumulation of a by-product and consumption of a nutrient. In still another embodiment, the cell is a cultured cell.

### BRIEF DESCRIPTION OF THE DRAWINGS

FIG. 1A-K illustrates the optical imprint (3D fluorescent spectra, X axis emission wavelength in nm, Y axis excitation wavelength in nm, Z axis fluorescence in RFU) of various potential biological marker such as A adenosine triphosphate or ATP, B flavin adenine dinucleotide or FAD, C histidine, D kinetin, E nicotinamide adenine dinucleotide or NADH, F phenylalanine, G pyroxidine, H riboflavin, I sanguinarine, J tryptamine and K tryptophane.

FIG. 2 illustrates an enlarged optical imprint (3D fluorescent spectra, X axis emission wavelength in nm, Y axis excitation wavelength in nm, Z axis fluorescence in RFU) of FIG. 1B (FAD).

FIG. 3 illustrates the 3D fluorescent spectrum (X axis emission wavelength in nm, Y axis excitation wavelength in nm, Z axis fluorescence in RFU) of fresh medium prior to its use in the cell culture.

FIG. 4A-C illustrates the 3D fluorescent spectra (X axis emission wavelength in nm, Y axis excitation wavelength in nm, Z axis fluorescence in RFU) of A a sample of a cell suspension (untreated) that has been cultured for 7 days, B the filtrate of the same cell suspension and C the resuspensed retentate of the same cell suspension.

FIG. 5A-F illustrates the fluorescence value (RFU) as a function of time (hours) for various potential biological markers. Column 1 represents the fluorescence values for the raw untreated sample. Column 2 represents the fluorescence values for the resuspended retentate. Column 3 represents the fluorescence values for the filtrate. Column 4 represents the fluorescent values for the corrected cells. Results are shown for fluorescent signals associated (λₑₓ, λₑₘ in nm) with A tryptamine (230,352), B phenylalanine-histidine (263,334), C pyridoxine (263,397), D tryptamine (275,349), E tryptophane (275,358), F NADH (275,448), G riboflavin-FAD (275,530), H ATP (300,400), I pyridoxine (323,394), J NADH (350,345), K sanguinarine (356,598), L Hoeschst-associated autofluorescence (360,465), M riboflavin (368,526), N FAD (368,532), O FAD (431,535) and P riboflavin-FAD (452,532).

FIG. 6A-F illustrates the offline values of a 7-day old cell culture obtained for A biomass concentration, B cell concentration, C and E rate of biomass accumulation, D and F rate of cellular proliferation. The rates of C and D are calculated from 5 experimental measures while the rates of E and F are calculated from 3 experimental measures.

FIG. 7A-B illustrates the results obtained with a 7-day old culture in function of time for A the offline value of biomass accumulation and B the fluorescent signal associated with tryptamine (230,352).

FIG. 8 illustrates the results obtained with a Principal Component Analisys (PCA) applied to culture variables and fluorescence signals. The proximity of variables indicates the level of correlation between them (i.e. very close variables on this graph are highly correlated). Results shown are those obtained for the cell suspension.

FIG. 9A-D illustrates correlations between the estimated biomass concentration (A an C) and the fluorescent signal associated with tryptamine (λₑₓ 230, λₑₘ 352) in the filtrate (A) or in the raw sample (C) as well as correlations between the estimated rate of cellular proliferation (B and D) and the fluorescent signal associated with riboflavin (λₑₘ 368, λₑₘ 526) in the resuspended retentate (B) or in the raw sample (D). Four different experiments have been compiled on this figure (Δ experiment 1, □ experiment 2, ∘ experiment 3, ⋄ experiment 4).

FIG. 10A-B illustrates representative results of a single experiment where the biomass concentration (A) or the rate of cellular proliferation (B) determined offline (⋄) are compared to the biomass concentration (A) or the rate of cellular proliferation (B) estimated with a linear regression technique (∘).

### DETAILED DESCRIPTION OF THE PREFERRED EMBODIMENT

In accordance with the present invention, there is provided a method for identifying endogenous fluorescent markers associated with a parameter of a cell.

Applicant herewith describes that a fluorescent marker associated with a cell that can be detected and that is linked to variation of a cell parameter. Accordingly, the method described herein broadly encompasses the correlation between an endogenous fluorescent signal obtained from a cell with a value for a parameter of the cell. One particular advantage of the present method is that the fluorescence that is measured is the autofluorescence of molecules endogenously present, produced (e.g. native) or consumed by the cells of the suspension. As such, there is no need, in this particular method, to add a fluorescent or fluorogenic compound to the sample of cells to be analyzed. There is also no need to genetically modify the cells to enable them to produce a fluorescent of fluorogenic compound.

Another advantage of the present method is, if a sample is retrieved from the cell culture, it does not need to be further processed before fluorescence quantification. For example, the cells do not need to be lysed or enzymatically treated prior to fluorescence quantification. However, to generate additional information, the sample can be filtered and the fluorescence quantification can be performed on the retentate (e.g. filtered cells that have been resuspended) and/or the filtrate. In this particular embodiment, the fluorescence obtained for the retentate will generate information about the fluorescence associated with the cells, whereas the fluorescence associated with the suspension (such as the culture medium) will generate information about the fluorescence that is not physically associated with the cells.

As used herein, the term “endogenous fluorescent marker” is a fluorescent signal (or a fluorescent value) associated with a cell that can be linked to a parameter of the cell. Endogenous fluorescent markers are linked to the presence of a molecule (or combination thereof) whose presence (or concentration) can be detected in the cell. As indicated above, the fluorescent marker is “endogenous” to the suspension of cells, because the fluorescent signal that is obtained is derived from the modulation of the concentration of the molecule in the cell and is not from the exogenous addition of fluorescent compounds. The terms “fluorescent signal” and “fluorescent value” are used herein interchangeably.

In an embodiment, the endogenous fluorescent marker is also associated with an endogenous biological marker of the cell, i.e. a marker that is biologically associated with the cell suspension. As such, the method does not contemplate identifying any fluorescent marker associated with the cell suspension, only those that are biologically linked to the cell.

In order to achieve this correlation, it is first determined if an optical imprint is associated with a fluorescent signal from the cell. This determination can be done as a function of time or as a function of a control cell. As used herein, the term “optical imprint” refers to the information of λ_{excitation}, λₑₘᵢₛₛᵢₒₙ couple(s) linked to the fluorescent signal (e.g. peak) or signals (e.g. peaks) associated with a specific molecule. Some molecules (such as ATP) have a single fluorescence associated peak and as such, their optical imprint will contain the wavelength information associated this single peak. Other molecules (such as FAD) have more than one fluorescence associated peaks and as such, their optical imprint will contain the wavelength information associated this numerous peaks.

In an embodiment, in order to achieve the correlation, it may be necessary to apply a correction to the fluorescence readings of the cell to compensate for a quenching effect of a cell culture additive, such as, for example, serum.

The detection of the optical imprint and of the fluorescent signal from the cell suspension can be made using various methods known in the art for the assessment of fluorescence. The optical imprint or fluorescent signal can be derived from a three-dimensional fluorescent spectra and/or a two-dimensional fluorescent spectra (e.g. a fluorescence reading at a selected excitation wavelength and/or at a selected emission wavelength). The optical imprint and fluorescent signal can be acquired from methods known in the art, for example, offline (e.g. spectrophotometer) or online (e.g. probe).

As used herein, the term “cell suspension” refer to a sample containing (or who contained) at least one cell that has been placed in a liquid (e.g. aqueous) medium. The cell suspension can be cultured in vitro or not. The cell suspension is either a sample of the suspension (such as a sample of a cell culture) or a derivative of such sample (such as, for example, a filtrate or resuspended retentate of a cell suspension). In an embodiment, derivatives of the cell suspension include, but are not limited to, cells present in the cell suspension (for example, cells that have been filtered out of the cell suspension) or a cell-free liquid obtained from the cell suspension.

In an embodiment, a single fluorescent signal as a function of time can be obtained from the cell suspension. For example, a fluorescent reading of a specific excitation and/or emission wavelength as a function of time can be obtained. In another embodiment, a plurality of fluorescent signals can be obtained from the cell suspension. For example, a three-dimensional fluorescent spectra (representation of the Relative Fluorescence Unit (RFU) in function of λ_{excitation} (in nm) and λₑₘᵢₛₛᵢₒₙ (in nm)) of the cell suspension can be obtained. As indicated above, the excitation and emission wavelengths can vary (for example by ±10 nm) between the type of culture that is being examined and the methods used to determine the fluorescence. Usually, the excitation wavelength of the cell suspension is between 203 and 750 nm and the emission wavelength reading ranges between 280 and 750 nm. The reading gain can be, for example, fixed at 130.

As indicated above, fluorescent signals of the cell suspension or its derivatives can be obtained. In an embodiment, the cell suspension is filtered and three-dimensional fluorescent spectra are obtained for the resuspended retentate and the filtrate as well. The RFU of the resuspended rententate can be corrected for the increase in cellular population with the ratio of the actual biomass concentration of the cell suspension in function of the initial biomass concentration in the cell suspension. For each 3D fluorescent spectra, fluorescent peaks can be identified and associated with a λ_{excitation}, λₑₘᵢₛₛᵢₒₙ couple. The autofluorescence measurement of the filtrate (e.g. culture medium) is related to molecules that are not necessarily physically with the cells (e.g. nutrients, by-products, waste products, etc.), whereas the retentate (e.g. cells resuspended in an appropriate aqueous solution such as saline) is related to the markers physically associated with the cell. This embodiment favors the comprehension of the ongoing metabolic phenomenon of the cell culture and facilitates the identification of the biological marker. When the sample is not treated prior to the measurement of its autofluorescence, the method allows the identification of biological markers that are directly tied to a culture parameter and as such would provide a platform for the design of a bioreactor probe. Biological markers can also be identified from filtrate or retentate autofluorescence and directly tied to a culture parameter. These biological markers can also be use for online monitoring, control and real time optimization when, as an example, combined with mathematical algorithms that generate extrapolation/prediction of the trend based on past samples.

Once it has been determined that the optical imprint is associated with a fluorescent signal of the cell suspension as a function of time, it is correlated to the values for the cell culture parameter (or more than one cell culture parameter). This correlation can be done with various mathematical means. In an embodiment, the comparison can be done graphically. For example, graphics can be compared visually in order to identify similar trends. This method can be suitable for a system where only a limited number of markers are studied. This assessment is qualitative and should ideally be followed by a numerical validation. In another embodiment, the comparison can be done numerically. For example, coefficient or covariance correlations can be calculated for each possible combination (potential biological marker—sample peaks). This method is particularly useful in systems where multiple potential biological markers are being studied. In another embodiment, the comparison can be a multi-variable analysis (e.g. a Principal Component Analysis for example). Multi-variables are used to identify correlations between potential biological markers and cell culture peaks. As an example, the Principal Component Analysis (PCA) enables the determination of correlation between the fluorescent values and the cell culture parameter by determining the proximity of variables. This technique is especially useful for the screening of correlations between any culture parameter and fluorescent values from any wavelength couple (excitation/emission) of a 3D spectrum. In still another embodiment, any other method allowing a rigorous discrimination between cell culture parameter is applicable to the identification of potential fluorescent marker.

This method described herein can be embodied in a screening system designed to perform the required steps. This screening system comprises at least two modules: a first module for performing the determination of the presence of the optical imprint of the endogenous marker in the cell suspension as a function of time and a second module for correlating the optical imprint with a parameter of the cell suspension. The first module can rely on a library of fluorescent optical imprints for various endogenous biological markers that are suspected to be present in the cell and compared them with a 2D or 3D spectra of fluorescence obtained from the cell as a function of time or as a function of a control. Once it has been established that the optical imprint of the endogenous marker is associated with a fluorescent signal as a function of time, then the correlation module relies on values provided for a specific cell parameter and determines if the optical imprint of the endogenous fluorescent marker is associated with the parameter of the cell suspension.

In another embodiment, the correlation step can use a processor or a correlation module. Once it has been established that the optical imprint of the endogenous marker is associated with a fluorescent signal as a function of time, then the processor or the correlation module relies on values provided for a specific cell suspension parameter and determines if the optical imprint of the endogenous fluorescent marker is associated with the parameter of the cell suspension.

As indicated above, the optical imprint of the endogenous fluorescent marker can be a single fluorescent signal (or peak) or a plurality of fluorescent signals (or peaks). As used herein, the term “endogenous biological marker” refers to molecules that are fluorescent, that are native (e.g. not exogenously added) to the cell that is present in the suspension and that are either produced or consumed by the cell during culture. In an embodiment, the fluorescent signal of the endogenous fluorescent marker can be derived from a three-dimensional (3D) fluorescent spectra of the endogenous biological marker. As used herein, the “3D fluorescent spectra” is a representation of the Relative Fluorescence Unit (RFU) in function of λ_{excitation} (in nm) and λₑₘᵢₛₛᵢₒₙ (in nm). For example, the 3D fluorescent spectra can be a plot where on the X axis the emission wavelength in nm is indicated, on the Y axis the excitation wavelength in nm is indicated, on the Z axis fluorescence the RFU is indicated. In an embodiment, a plurality of fluorescent signals for a single endogenous biological marker is obtained. In another embodiment, fluorescent signals for a plurality of endogenous biological markers are obtained to generate a library of fluorescent signals of the markers. In yet another embodiment, for each potential biological markers, a unique three-dimensional spectra (λ_{excitation}, λₑₘᵢₛₛᵢₒₙ, Relative Fluorescence Unit (RFU)) can be then obtained. Usually, the excitation wavelength of each potential biological marker is between 203 and 750 nm and the emission wavelength reading ranges between 280 and 750 nm. The reading gain can be fixed at 130. This three-dimensional spectra will be referred to as the optical imprint of potential biological marker. For each optical imprint, fluorescent peaks are identified and associated with a λ_{excitation}, λₑₘᵢₛₛᵢₒₙ couple. In another embodiment, the fluorescent signal of the endogenous biological marker can be a value obtained from a reading at a selected excitation and/or emission wavelength.

If a 3D fluorescent spectra is generated for the potential endogenous biological markers, fluorescent peaks are identified and compared with peaks from other potential biological marker and classified as “unique” or “shared”. As used herein, a “unique” peak refers to a peak that is only found in the optical imprint of one potential biological marker. Thus, a “unique” peak is not redundant between potential biological markers. Its summit is clearly independent and distinct from all the other peaks. These “unique” peaks facilitate the discrimination of different potential biological marker in the more complex three-dimensional spectra of the cell suspension. On the other hand, a “shared” peak is found in more than one optical imprint of the potential biological marker. It is thus somehow redundant between at least two optical imprints. Although the “shared” peak could also provide valuable information on the status of the cell culture, one cannot identify a single potential biological marker to this specific peak. The excitation and emission wavelengths of the fluorescent signal can vary (for example by ±10 nm) between the type of culture that is being examined and the methods used to determine the fluorescence.

In still another embodiment, the fluorescent signal of the cell suspension can be obtained offline (e.g. by sampling the culture and/or processing the sample prior to fluorescent determination) with the use of a spectrophotometer or online (e.g. by using a probe).

In a further embodiment, the optical imprint of the endogenous fluorescent marker is the fluorescent signal of the chemically purified form of the endogenous fluorescent marker (e.g. more than 95% or 99% pure).

The method described herein can be used with any cells that can be cultured in vitro, such as prokaryotic (bacteria) and eukaryotic cells (yeasts, molds, plant, algae, animal, insect, etc.). This method can also be used to monitor the growth of prions, viruses or phages that are cultured in vitro and have infected an appropriate host cell. The cells cultured can be derived from a single type or genera of cells or they can be a mixture of more than one cell type or genera.

Even though the methods described herein do not rely on the use of genetic engineering to produce a fluorescent signal, cells that have been genetically modified or chemically mutated can also be used. The methods provided herewith will enable the determination of various parameters associated with their culture and will enable the optimization of their culture.

Fluorescence can be quantified by various means in the art. Fluorescence in several wavelengths can be detected by an array detector, to detect compounds from HPLC flow. Also, thin layer chromatography plates and microscopy can also be used to visualize the endogenous biological marker. Ideally, the fluorescence of a sample is determined rapidly and accurately with a spectrophotometer.

A further advantage of this method is that it can be used on static (batch) culture, fed-batch culture, continuous or perfused culture. The term “continuous culture” refers to the growth of cells in culture medium in a culture chamber, whereby fresh medium or elements thereof is added while suspension is partially removed. Medium adding rate and suspension removal rate are generally the same so as to keep the culture volume approximately constant. “Perfused culture” refers to the growth of cells in culture medium in a culture chamber, whereby fresh medium or elements thereof is added while culture medium is partially removed whereas cells are retained in the culture chamber. Fresh medium adding rate and culture medium removal rate are generally the same so as to keep the culture volume approximately constant. On the contrary, the term “static culture” refers to the growth of cells for a definite period of time where the cells and medium are both recuperated at the end of the incubation and where no additional medium is added during the culture and no suspension is removed. Further, the term “fed-batch culture” refers to an hybrid of the previous two culture methods, where, during the culture, some fresh medium or elements thereof is added to the culture. However, in fed-batch culture, no suspension is removed. Methods for growing plant tissue culture cells are known to those of skill in the art.

In an embodiment, the parameter that is determined by the method is the biomass concentration. As used herein, the term “biomass concentration” the grams of dry weight of cells per liter of culture. The dry weight of cells can easily be determined by those skilled in the art by placing a sample containing cells from the culture onto pre-weighed container, removing the culture media, drying the cells, and weighing them. This cell culture parameter is important because it generates information about the amount of cells in the culture. If the biomass increases during time, it is assumed that the cells accumulate and biosynthesize metabolites meaning cells metabolize properly and are in a growth phase.

In an embodiment, the parameter that is determined by the method is the rate of biomass accumulation (h⁻¹). As used herein, the term “rate of biomass accumulation” is used as related to the length of time required for the biomass (dry weight of cells) to double. This cell culture parameter is important because it generates information about the amount of cells in the culture. If the rate of biomass accumulation remains positive during time, it is assumed that the cells accumulate and biosynthesize metabolites meaning cells metabolize properly and are in a growth phase.

Another parameter that can be easily assessed by the methods described herein is the cellular concentration, i.e. the number of cells per L of medium. Cellular concentration is routinely determined by those skilled in the art by using routine techniques (hematocytometer, cell counting, FACS, etc.). This cell culture parameter is important because it generates information about the amount of cells in the culture. If the cellular concentration increases during time, it is assumed that the cells accumulate and biosynthesize metabolites meaning cells metabolize properly and are in a growth phase.

A further parameter that can be easily assessed by the methods described herein is the rate of cellular proliferation (h⁻¹). The rate of cellular proliferation is used herein as related to the length of time required for a cell go through an full cellular cycle (e.g. from the moment division until the next division). This cell culture parameter is important because it generates information about the amount of cells in the culture. If the rate of cellular proliferation remains positive during time, it is assumed that the cells accumulate and biosynthesize metabolites meaning cells metabolize properly and are in a growth phase.

Markers for additional cell culture parameters can be obtained using this method, as long as the additional cell culture parameter is associated with a detectable fluorescent signal. These additional markers include, but are not limited to, biomass accumulation, accumulation of a by-product and consumption of nutrients.

Because the methods described herein generate valuable endogenous fluorescent markers, the present application also provides a method of estimating a parameter of a cell from a fluorescence signal of at least one endogenous marker. The first step in this method is the identification of the endogenous fluorescent marker with the method described herein. A mathematical relationship between the fluorescent signal(s) of the endogenous marker and of the cell parameter is then obtained. This can be done, for example, by obtaining a fluorescent signal(s) for the endogenous marker as a function of time and comparing it to a value for the cell culture parameter as a function of time. The mathematical relationship can then be used to estimate the parameter value based on the fluorescent signal of the endogenous marker. Any mathematical relationship can be used, such as, for example, a linear regression and/or multiple variable analysis. When linear regression is used, the slope of the linear regression is useful in the estimation of the cell culture parameter. Any cell culture parameter described herein can be used in this method.

The present invention will be more readily understood by referring to the following examples which are given to illustrate the invention rather than to limit its scope.

### EXAMPLE I

### Preparation of a Plurality of Optical Imprints of Potential Biological Markers

Calibration of the spectrophotometer. For each candidate marker, a 500 μM aqueous solution was prepared with pure chemicals. Each sample of candidate marker was scanned in 3D (λ_{excitation}, λₑₘᵢₛₛᵢₒₙ, Relative Fluorescence Unit or RFU) using the Saphire^{2™} spectrophotometer (Tecan) at excitation and emission wavelengths starting from 50 nm under the theoretical wavelengths of the marker-candidate (i.e. excitation and emission wavelengths associated to the peak from the literature) to 50 nm over the theoretical wavelengths. If the reading of the signal is over, the reading gain was adjusted or the original solution was diluted until a clear signal was obtained. This enables the identification of the excitation and emission wavelength corresponding to the maximal amplitude reading for each of the candidate marker.

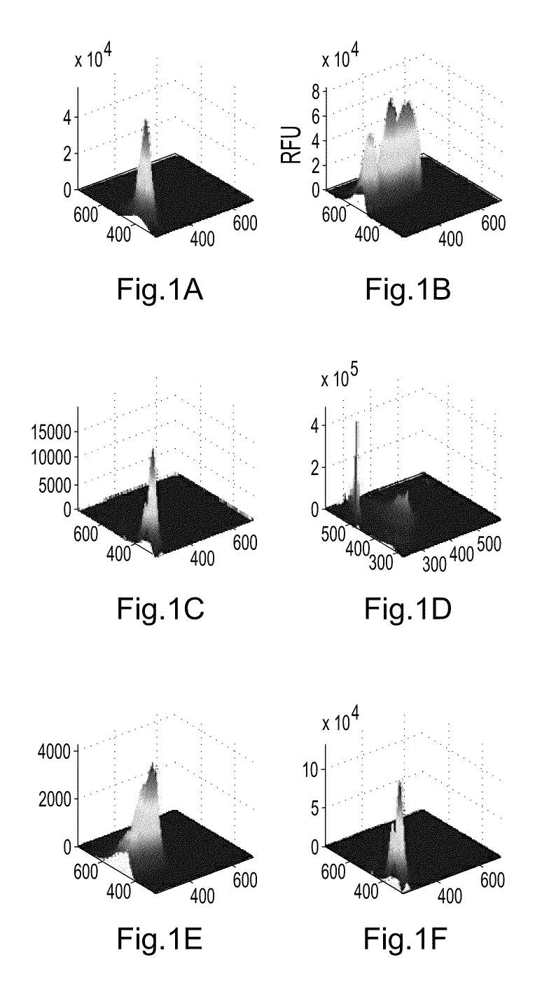

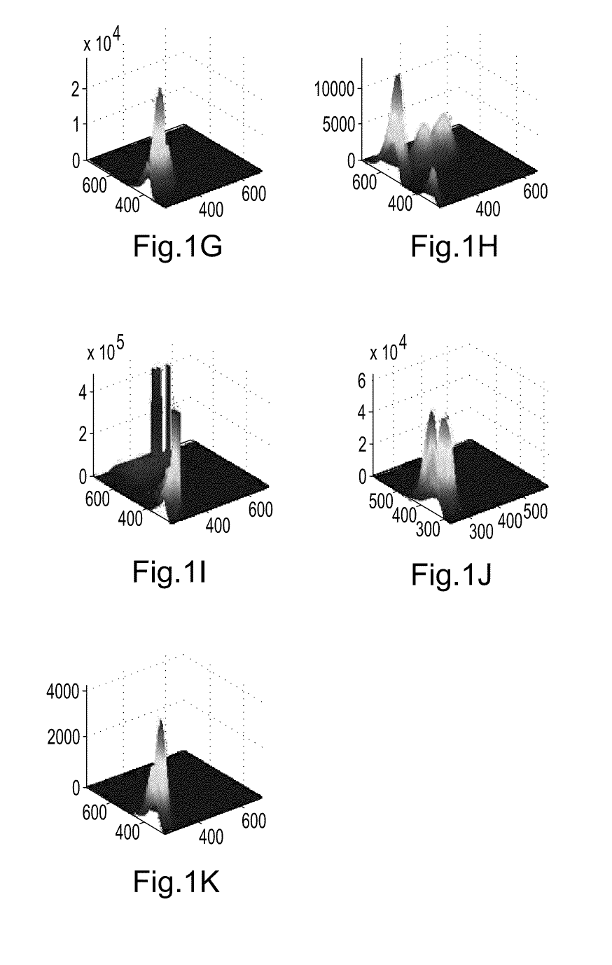

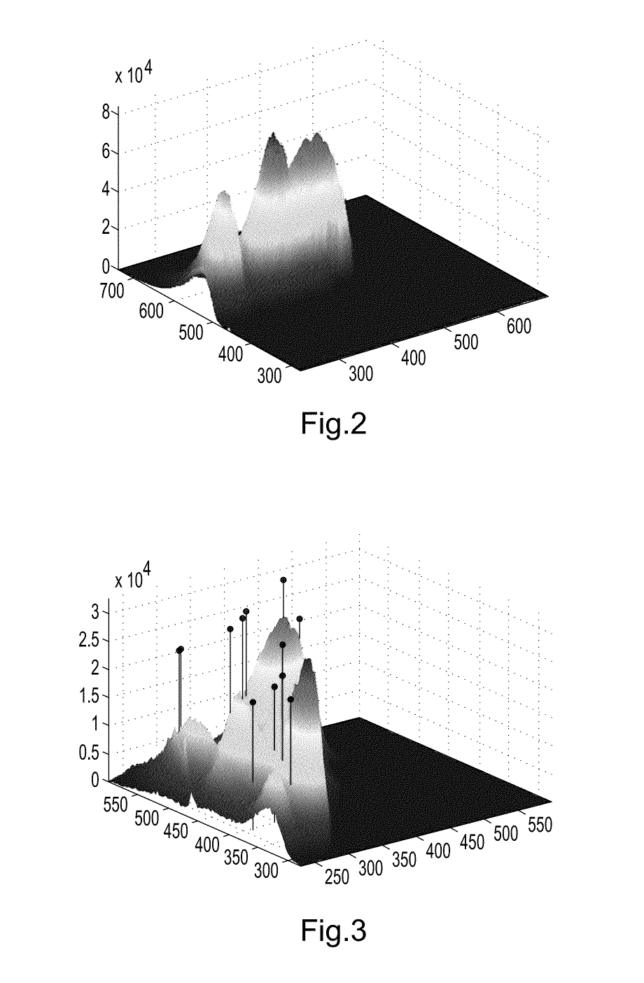

Optical imprint. The solutions of the candidate markers were also scanned in 3D (λ_{excitation}, λₑₘᵢₛₛᵢₒₙ, Relative Fluorescence Unit or RFU) using the Saphire^{2™} spectrophotometer (Tecan) at the appropriate gain for excitation (e.g. 130), excitation wavelengths from 230 to 750 nm with a minimal step and emission wavelengths from 230 to 750 nm with a minimal step. This 3D scan is also called the optical imprint of the candidate marker. The parameters for the spectrophotometer were the following: bandwidth 5 nm, number of repetition per measurements 5, integration time 41 μs, lag time 0 μs, Z position 11019 μm. The optical imprints of various potential biological markers are shown in FIG. 1. An enlarged representation of the optical imprint of FAD is shown in FIG. 2.

Determination of couples of excitation and emission wavelength. The optical imprints of several potential biological markers have been obtained as previously described. Tables 1 and 2 summarizes the peaks that have been identified for some potential biological markers.

TABLE 1
Peaks excitation and emission wavelength (in
nm) for some potential biological markers.

| Potential | |
|---|---|
| biological | |
| marker | (λ_{excitation}, λₑₘᵢₛₛᵢₒₙ) |
| ATP | (300, 400) |
| FAD | (275, 530) (368, 532) (431, 535) |
| | (452, 532) |
| Histidine | (263, 334) |
| NAD(P)H | (275, 448) (350, 445) |
| Pyridoxin | (263, 397) (323, 394) |
| Phenylalanin | (263, 334) |
| Riboflavin | (275, 530) (368, 526) (452, 532) |
| Sanguinarin | (356, 598) |
| Tryptamine | (230, 352) (275, 349) |
| Tryptophane | (275, 358) |

TABLE 2
Unique and shared peaks for the various
potential biological markers.

| Unique peaks | | Shared peaks | |
|---|---|---|---|
| (λ_{excitation}, | Potential | (λ_{excitation}, | Combination of poten- |
| λₑₘᵢₛₛᵢₒₙ) | biological marker | λₑₘᵢₛₛᵢₒₙ) | tial biological markers |
| 230, 352 | Tryptamine | 263, 334 | Histidine, Phenlylalanin |
| 263, 397 | Pyridoxin | 275, 530 | FAD, Riboflavin |
| 275, 349 | Tryptamine | 452, 532 | FAD, Riboflavin |
| 275, 358 | Trytptophan | | |
| 275, 448 | NADH | | |
| 300, 400 | ATP | | |
| 323, 394 | Pyridoxin | | |
| 350, 445 | NADH | | |
| 356, 598 | Sanguinarin | | |
| 368, 526 | Riboflavin | | |
| 368, 532 | FAD | | |
| 431, 535 | FAD | | |

### EXAMPLE II

### Cell Culture and 3D Spectrophotometric Fluorescent Spectra Associated Thereto

Cell culture. In order to prepare the B5 medium, 500 mL water was poured in a graduated cylinder and stirred. Then, sequentially, 15 mL of a B5 macro-nutrients solution, 1.5 mL of a CaCl₂ (1 M), 1.5 mL of B5 micro-nutrients solution, 1.5 mL of EDTA-Fe solution (100 mM), 1.5 mL of B5 vitamins, 0.3 mL of a 2,4-D solution (4.4 mM), 0.15 mL of a kinetin solution (4.65 mM) and 45 g of sucrose were added to the stirring water. The volume of the solution was adjusted to 1 L with water and the solution was stirred to obtain the dissolution of the chemicals. The pH of the solution was then adjusted to 5.5 using a 1M KOH solution. The solution was then sterilized (autoclaved 15 min, 121° C. and 15 psig) for use in the cell culture. In order to prepare the cells, seeds of Eschscholtzia californica (Richters inc, #S4720) were sterilized (2 min in EtOH 70%; 15 min in Javex; washed twice using sterile water; placed on 2 wet papers within a parafilm sealed sterile Petri dish). Callus were then induced by transferring the germs issued from seeds on solid B5 medium supplemented with 2 g/L Phytogel™. Callus were maintained and transferred to fresh solid B5 medium every 1 to 3 months depending on growth. The cell line was initiated by transferring the callus cells into liquid B5 medium in a shake flask. Every 14 days, 30 mL of a two-week old cell suspension was added to 60 mL of fresh medium. The cells were cultured for 14 days during constant agitation (120 RPM) at 25° C. Aliquots were taken at various intervals.

3D spectrophotometric fluorescent spectra of the cell culture. Samples of the cell culture were scanned in 3D (λ_{excitation}, λₑₘᵢₛₛᵢₒₙ, Relative Fluorescence Unit or RFU) using the Saphire^{2™} spectrophotometer (Tecan) at the appropriate gain for excitation (e.g. 130), excitation wavelengths from 230 to 750 nm with a minimal step and emission wavelengths from 230 to 750 nm with a minimal step. The parameters for the spectrophotometer were the following: bandwidth 5 nm, number of repetition per measurements 5, integration time 41 μs, lag time 0 μs, Z position 11019 μm. A representative 3D fluorescent spectra of a fresh medium (prior to its use in the cell culture) is shown in FIG. 3. On that figure, the lines ending with a closed circle show the localization of the potential biological markers characterized in Example 1.

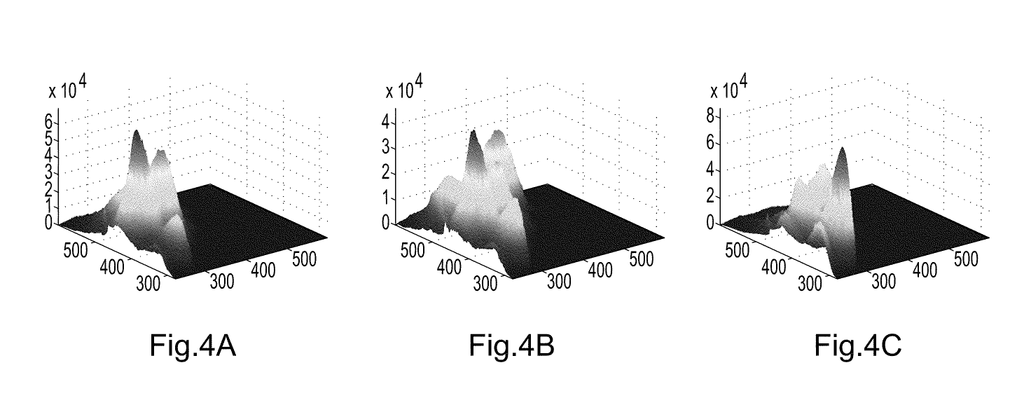

Analysis of fluorescent parameters of samples cell culture. Raw samples of 200 μL were transferred into an opaque black spectrophotometric plate. Raw samples were also filtered (using a 0.45 μM filter) and 150 mL of the filtered samples were also transferred into the opaque black spectrophotometric plate. The retentate was resuspended in 8.25% saline solution and transferred into the opaque black spectrophotometric plate. 2D and 3D scans of the raw samples, filtered samples and resuspended rententate were performed as indicated above. The 3D scan enabled the determination of RFU for each (λ_{excitation}, λₑₘᵢₛₛᵢₒₙ) couples. 3D spectrophotometric fluorescent spectra of samples of a 7-day old cell culture have been obtained and the results are shown in FIG. 4.

Determination of RFU amplitude. The RFU amplitude of the raw sample, the filtrate and the resuspended retentate has been obtained by direct measurement using a spectrophotometer. Results are shown in Table 3.

TABLE 3
RFU amplitude obtained for various potential biological markers

| | Potential | Raw | | |
|---|---|---|---|---|
| | biological | sample | Filtrate | Retentate |
| (λₑₓ, λₑₘ) in nm | marker | (RFU) | (RFU) | (RFU) |
| 230, 352 | Tryptamine | 13 077 | 26 211 | 6 705 |
| 263, 397 | Pyridoxine | 13 689 | 28 008 | 12 356 |
| 263, 334 | Histidine, | 20 509 | 20 100 | 14 378 |
| | Phenlylalanie | | | |
| 275, 349 | Tryptamine | 34 956 | 66 126 | 26 895 |
| 275, 358 | Trytptophan | 28 063 | 53 322 | 22 281 |
| 275, 448 | NADH | 53 652 | 51 308 | 40 807 |
| 275, 530 | FAD, Riboflavine | 10 778 | 11 183 | 15 752 |
| 300, 400 | ATP | 24 248 | 32 971 | 17 558 |
| 323, 394 | Pyridoxine | 17 356 | 23 552 | 12 475 |
| 350, 445 | NADH | 42 585 | 87 491 | 25 205 |
| 356, 598 | Sanguinarine | 1 920 | 4 747 | 1 791 |
| 368, 526 | Riboflavin | 9 859 | 20 497 | 19 591 |
| 368, 532 | FAD | 8 449 | 17 598 | 17 560 |
| 431, 535 | FAD | 3 951 | 1 429 | 12 700 |
| 452, 532 | FAD, Riboflavin | 2 162 | 1 036 | 6 196 |

Corrected values have been calculated from the retentate value so as to be representative of the signal amplitude that would result from a reading of the cells fluorescence at the actual cell concentration within the original culture. In order to do so, the raw RFU data of the retentate is multiplied by the ratio of the actual biomass concentration within the original culture/biomass concentration of suspended cells for fluorescence reading with the spectrophotometer. The corrected RFU amplitude is representative of the increase in cellular population occurring during culture. Results are shown in the last column of Table 4.

TABLE 4
RFU amplitude (including corrected biomass RFU) obtained
for various potential biological markers.

| | Potential | Raw | | | Corrected |
|---|---|---|---|---|---|
| (λₑₓ, λₑₘ) | biological | sample | Filtrate | Retentate | cells |
| in nm | marker | (RFU) | (RFU) | (RFU) | (RFU) |
| 230, 352 | Tryptamine | 13 077 | 26 211 | 6 705 | 23 402 |
| 263, 397 | Pyridoxin | 13 689 | 28 008 | 12 356 | 25 007 |
| 263, 334 | Histidine, | 20 509 | 20 100 | 14 378 | 17 946 |
| | Phenlylalanie | | | | |
| 275, 349 | Tryptamine | 34 956 | 66 126 | 26 895 | 59 041 |
| 275, 358 | Trytptophane | 28 063 | 53 322 | 22 281 | 47 608 |
| 275, 448 | NADH | 53 652 | 51 308 | 40 807 | 45 810 |
| 275, 530 | FAD, Riboflavin | 10 778 | 11 183 | 15 752 | 9 984 |
| 300, 400 | ATP | 24 248 | 32 971 | 17 558 | 29 438 |
| 323, 394 | Pyridoxine | 17 356 | 23 552 | 12 475 | 21 028 |
| 350, 445 | NADH | 42 585 | 87 491 | 25 205 | 78 116 |
| 356, 598 | Sanguinarine | 1 920 | 4 747 | 1 791 | 4 238 |
| 368, 526 | Riboflavine | 9 859 | 20 497 | 19 591 | 18 300 |
| 368, 532 | FAD | 8 449 | 17 598 | 17 560 | 15 712 |
| 431, 535 | FAD | 3 951 | 1 429 | 12 700 | 1 275 |
| 452, 532 | FAD, Riboflavin | 2 162 | 1 036 | 6 196 | 925 |

### EXAMPLE III

### Determination of Biological Markers

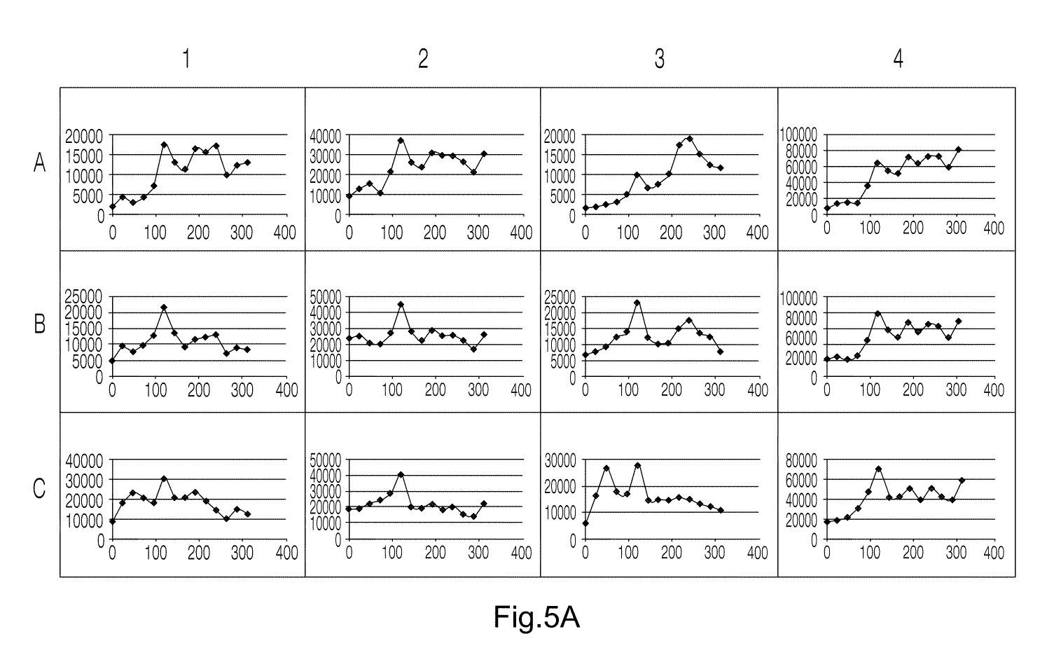

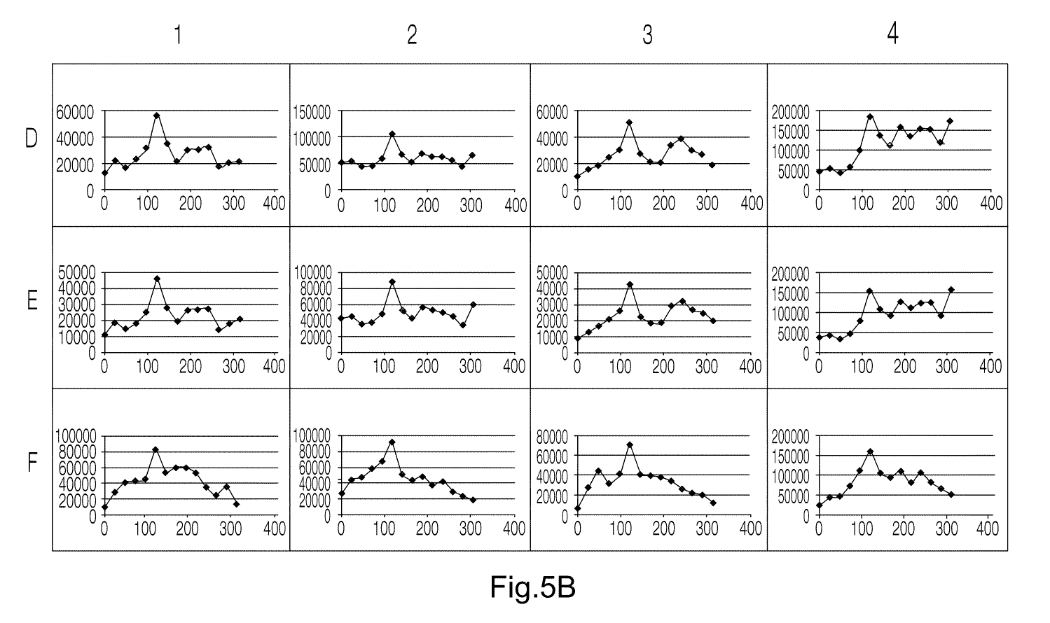

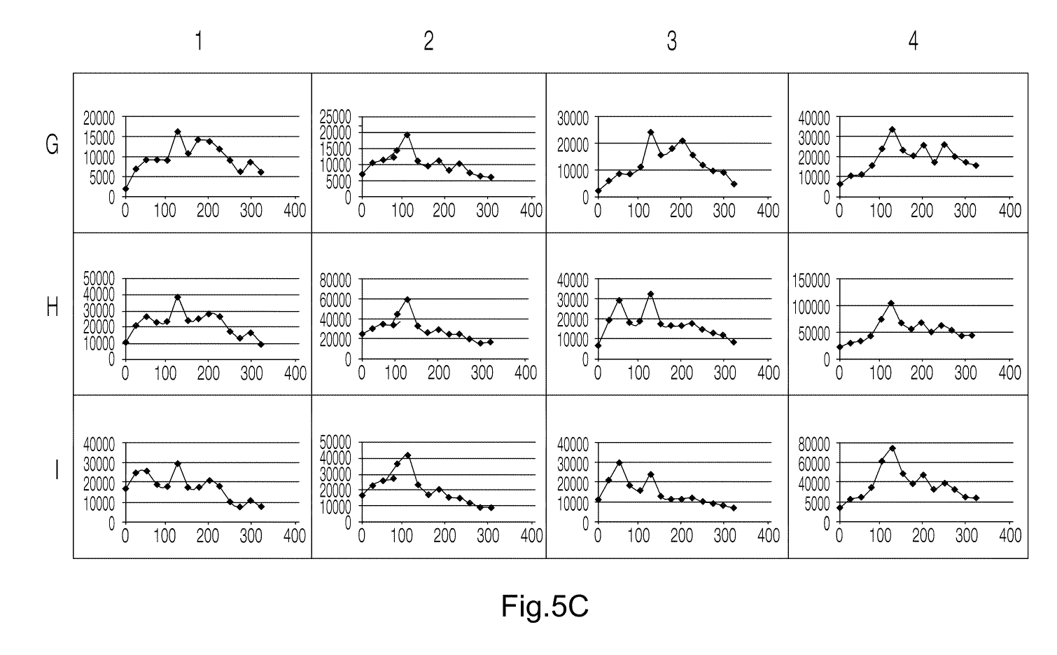

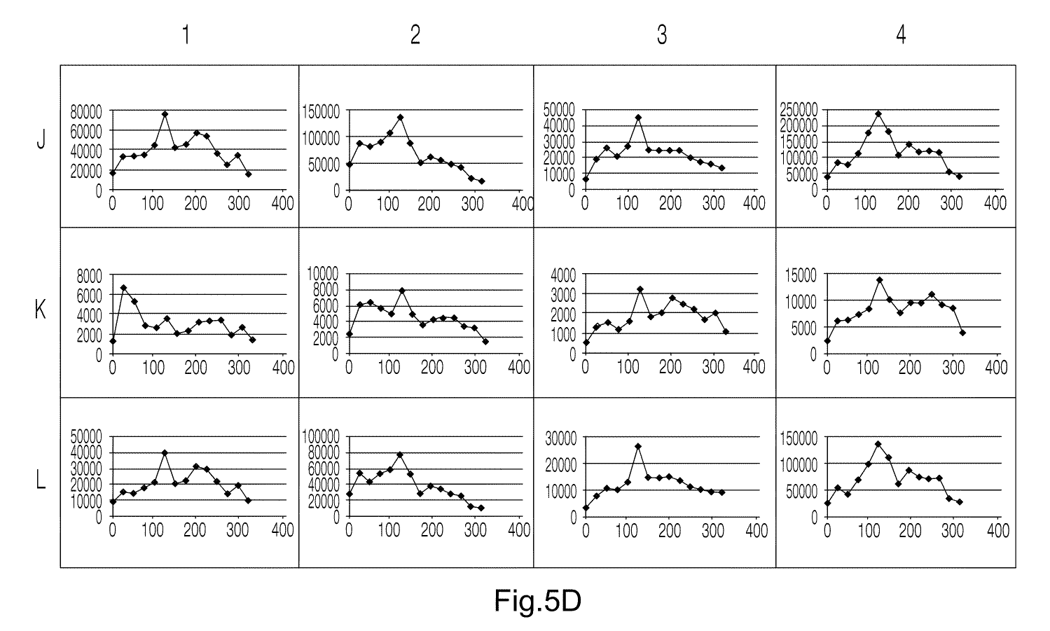

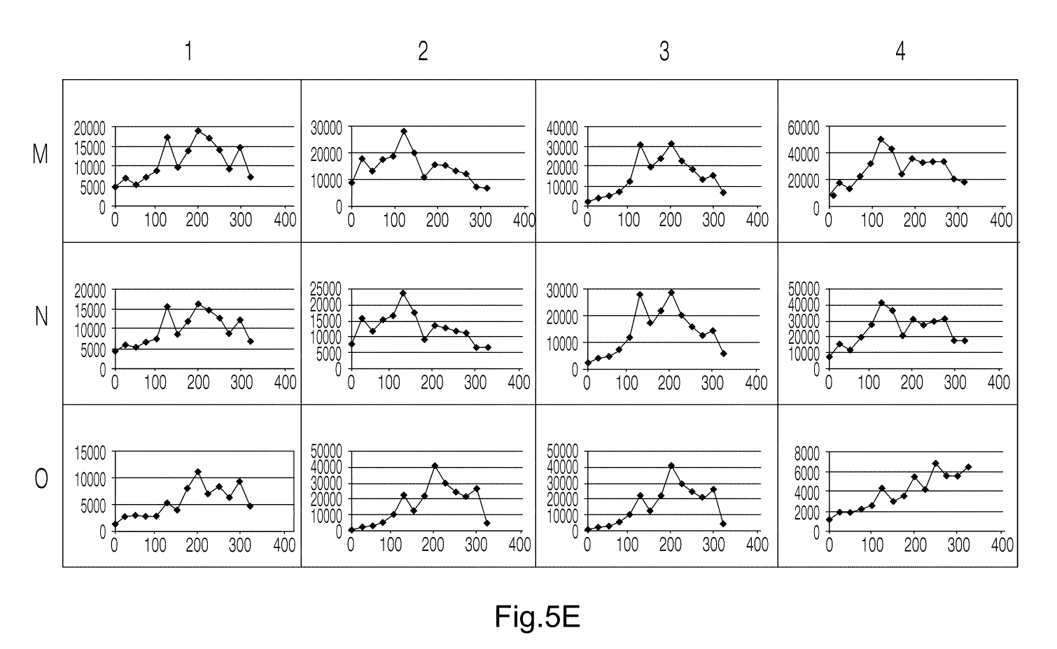

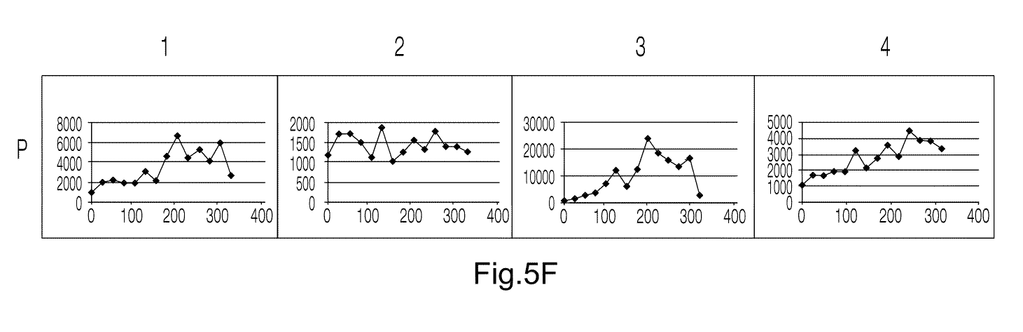

Spectrophotometric profiles of potential biological markers. Four spectrophotometric profiles of the raw sample, the filtered sample, the resuspended retentate and the culture medium as a function of time were obtained for every cell culture sample. Results are shown in FIG. 5 for various potential biological markers.

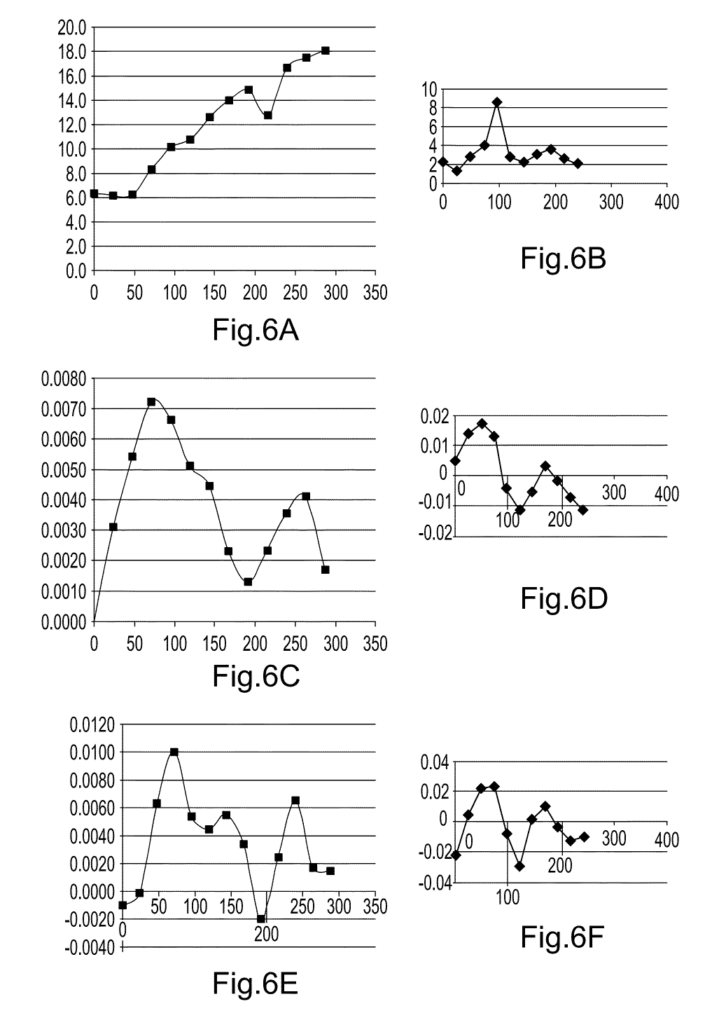

Determination of the offline biomass concentration. An empty centrifugal tube and a foil paper were first weighted independently. A 2.5 mL aliquot of well mixed cell suspension was taken from the culture and placed into a centrifugal tube using a sterile pipette. The centrifugal tube containing the cell suspension was then weighted. The sample was transferred into a syringe and filtered using a 0.45 μm nitrocellulose filter. The retentate (the cells) was transferred onto the weighted foil paper and was dried in an oven at 80° C. overnight. The foil paper was then weighted. The biomass concentration was obtained by dividing the dry biomass weight (foil and cell weight after oven minus foil weight) by the sample weight (centrifugal tube with sample weight minus centrifugal tube weight). Results obtained for the 7-day old culture are shown in FIG. 6A.

Determination of the offline cell concentration and cellular proliferation rate. A 0.5 mL sample of a well mixed cell suspension was retrieved under sterile conditions and placed in a microcentrifuge tube. Then, 1 mL of an enzymatic solution (citrate buffer (200 mM, pH 4.5) supplemented with sucrose (60 g/L), cellulase (10 U/mL, Sigma-Aldrich. C1184), hemi-cellulase (0.03 U/mL, Sigma-Aldrich. H2125) and pectinase (0.2 U/mL, Sigma-Aldrich. P5146) was added to the microcentrifuge tube. The microcentrifuge tube was placed horizontally on an orbital shaker (120 RPM) for 1 h. The suspension was aspirated three times using a 1 mL micropipette or a cut disposable tip. The microcentrifuge tube was placed horizontally on an orbital shaker (120 RPM) for 30 min. The suspension was aspirated three times using a 1 mL micropipette or a cut disposable tip. To a 50 μL sample of the suspension, 200 μL of a Carbol fushin solution was added (Kao K N (1982) Staining methods for protoplasts and cells. In: Wetter L R, Constabel F (eds) Plant tissue culture methods, 2nd edn. National Research Council of Canada, Saskatoon, Canada, pp 67-71). The suspension was then transferred onto an hemacytometer (Hausser Scientific. 3720) for cell counting under normal light conditions using a microscope. The cell concentration was obtained by dividing the number of cells by the counting chamber volume while taking dilutions into account. The cell proliferation rate was obtained for two consecutive samples by the evaluation of:

[ln(c₂/c₁)/(t₂−t₁)]

where c₁ and c₂ are the cell concentration of the first and second samples while t₁ and t₂ are the sampling time of these samples. Statistical analysis of the results obtained are described in Sirois J. (2000) Ph.D. thesis, Ecole Polytechnique de Montreal. Results obtained for the 7-day old culture are shown in FIG. 6B to 6F.

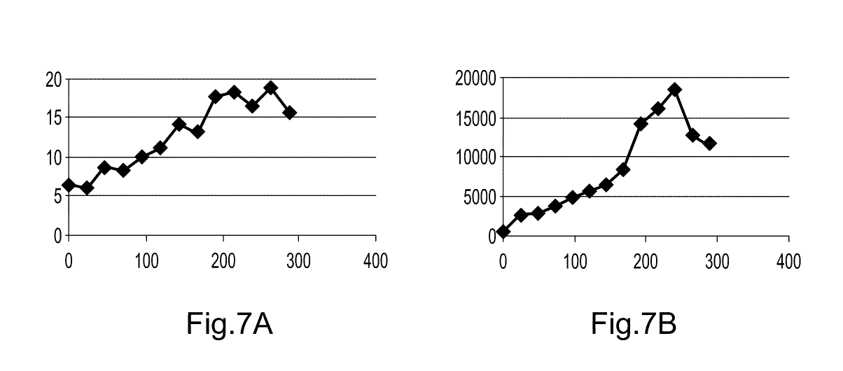

Identification of biological markers with visual inspection. The profiles obtained for the potential biological markers are visually compared for similar profiles of the offline measures obtained. As it can be seen in FIG. 7, the biomass concentration profile in function of time is similar to the tryptamine profile (230,352) as a function of time in the filtrate. As such, it can be assumed that the fluorescent signal associated with tryptamine (230,352) is indicative of biomass accumulation in the cell culture.

Identification of biological markers with covariance correlations. For a specific cell culture parameter (e.g. biomass concentration), the results of fluorescence of a potential biological markers of four independent experiments were compiled. Statistical significance is determined based on the covariance correlations. Table 5 and 6 show the results obtained with this technique for biomass accumulation. In Table 6, the highlighted cells indicate statistical significance, i.e. that the marker is useful to predict biomass concentration.

TABLE 5
Regression coefficients for various endogenous biological markers.

| | | | Regression coefficients (r²) | | | | | | | |
|---|---|---|---|---|---|---|---|---|---|---|
| | | | Culture #1 | | Culture #2 | | Culture #3 | | Culture #4 | |
| Biomass | | | Growth | Entire cult. | Growth | Entire cult. | Growth | Entire cult. | Growth | Entire cult. |
| 230/352 | Tryptamine | Untreated | 0.726 | 0.612 | 0.896 | 0.829 | 0.879 | 0.869 | 0.708 | 0.659 |
| | | Retentate | 0.643 | 0.474 | 0.820 | 0.838 | 0.916 | 0.921 | 0.748 | 0.644 |
| | | Filtrate | 0.832 | 0.704 | 0.929 | 0.776 | 0.956 | 0.917 | 0.860 | 0.535 |
| | | Corrected cells | 0.889 | 0.620 | 0.960 | 0.935 | 0.967 | 0.968 | 0.906 | 0.845 |
| 263/334 | Phenlylalanie | Untreated | 0.134 | 0.185 | 0.458 | 0.154 | −0.045 | −0.159 | 0.564 | 0.321 |
| | Histidine | Retentate | −0.066 | −0.069 | 0.690 | 0.491 | 0.624 | 0.494 | 0.820 | 0.650 |
| | | Filtrate | 0.378 | 0.451 | 0.518 | 0.084 | 0.257 | 0.190 | 0.626 | 0.311 |
| | | Corrected cells | 0.732 | 0.528 | 0.977 | 0.895 | 0.946 | 0.923 | 0.932 | 0.888 |
| 263/397 | Pyridoxine | Untreated | −0.186 | −0.055 | 0.672 | 0.388 | 0.265 | 0.002 | 0.582 | 0.492 |
| | | Retentate | −0.307 | −0.292 | −0.031 | 0.004 | 0.045 | −0.172 | 0.320 | 0.281 |
| | | Filtrate | −0.271 | −0.142 | 0.544 | 0.167 | 0.081 | −0.118 | 0.371 | 0.057 |
| | | Corrected cells | 0.572 | 0.360 | 0.948 | 0.895 | 0.957 | 0.932 | 0.945 | 0.901 |
| 275/349 | Tryptamine | Untreated | 0.128 | 0.171 | 0.447 | 0.140 | 0.055 | −0.079 | 0.572 | 0.339 |
| | | Retentate | 0.087 | 0.040 | 0.608 | 0.493 | 0.648 | 0.558 | 0.724 | 0.596 |
| | | Filtrate | 0.423 | 0.461 | 0.634 | 0.379 | 0.334 | 0.320 | 0.674 | 0.440 |
| | | Corrected cells | 0.754 | 0.525 | 0.978 | 0.893 | 0.950 | 0.931 | 0.914 | 0.869 |
| 275/358 | Trytptophane | Untreated | 0.174 | 0.182 | 0.508 | 0.312 | 0.138 | −0.017 | 0.610 | 0.391 |
| | | Retentate | 0.055 | −0.035 | 0.605 | 0.539 | 0.674 | 0.581 | 0.767 | 0.575 |
| | | Filtrate | 0.472 | 0.467 | 0.616 | 0.472 | 0.419 | 0.417 | 0.690 | 0.479 |
| | | Corrected cells | 0.742 | 0.453 | 0.981 | 0.910 | 0.952 | 0.935 | 0.926 | 0.870 |
| 275/448 | NADH | Untreated | 0.159 | 0.296 | 0.833 | 0.438 | 0.725 | 0.504 | 0.685 | 0.645 |
| | | Retentate | −0.294 | −0.095 | 0.265 | −0.172 | 0.202 | −0.116 | 0.549 | 0.318 |
| | | Filtrate | −0.098 | 0.059 | 0.720 | 0.241 | 0.592 | 0.318 | 0.642 | 0.318 |
| | | Corrected cells | 0.420 | 0.473 | 0.942 | 0.634 | 0.926 | 0.847 | 0.943 | 0.797 |
| 275/530 | Riboflavine | Untreated | 0.273 | 0.346 | 0.772 | 0.584 | 0.785 | 0.619 | 0.645 | 0.671 |
| | FAD | Retentate | −0.309 | −0.127 | 0.308 | −0.133 | 0.058 | −0.209 | −0.182 | 0.229 |
| | | Filtrate | 0.392 | 0.469 | 0.931 | 0.623 | 0.837 | 0.774 | 0.845 | 0.714 |
| | | Corrected cells | 0.539 | 0.543 | 0.965 | 0.717 | 0.962 | 0.921 | 0.915 | 0.845 |
| 300/400 | ATP | Untreated | −0.150 | 0.053 | 0.702 | 0.304 | 0.489 | 0.242 | 0.563 | 0.494 |
| | | Retentate | −0.445 | −0.257 | 0.334 | −0.076 | 0.049 | −0.251 | 0.437 | 0.350 |
| | | Filtrate | −0.332 | −0.141 | 0.565 | 0.106 | 0.166 | −0.049 | 0.387 | 0.086 |
| | | Corrected cells | 0.369 | 0.389 | 0.964 | 0.739 | 0.950 | 0.873 | 0.952 | 0.848 |
| 323/394 | Pyridoxine | Untreated | −0.710 | −0.433 | −0.088 | −0.347 | −0.312 | −0.470 | −0.097 | −0.110 |
| | | Retentate | −0.526 | −0.315 | 0.060 | −0.288 | −0.311 | −0.514 | −0.062 | −0.092 |
| | | Filtrate | −0.725 | −0.501 | 0.010 | −0.482 | −0.435 | −0.576 | −0.037 | −0.266 |
| | | Corrected cells | 0.169 | 0.242 | 0.928 | 0.656 | 0.870 | 0.728 | 0.972 | 0.657 |
| 350/445 | NADH | Untreated | 0.156 | 0.297 | 0.675 | 0.286 | 0.604 | 0.458 | 0.602 | 0.583 |
| | | Retentate | −0.546 | −0.282 | 0.365 | −0.243 | 0.206 | −0.147 | 0.053 | −0.058 |
| | | Filtrate | −0.011 | 0.102 | 0.708 | 0.473 | 0.587 | 0.429 | 0.484 | 0.290 |
| | | Corrected cells | 0.124 | 0.254 | 0.953 | 0.332 | 0.917 | 0.789 | 0.937 | 0.502 |
| 356/598 | Sanguinarine | Untreated | −0.431 | −0.253 | −0.336 | −0.303 | −0.396 | −0.334 | −0.616 | 0.158 |
| | | Retentate | −0.453 | −0.159 | 0.139 | −0.311 | −0.014 | −0.307 | 0.035 | 0.022 |
| | | Filtrate | 0.519 | 0.574 | 0.736 | 0.655 | 0.800 | 0.854 | 0.840 | 0.658 |
| | | Corrected cells | 0.557 | 0.628 | 0.908 | 0.302 | 0.944 | 0.855 | 0.961 | 0.557 |
| 360/465 | Autofluorescence | Untreated | 0.299 | 0.395 | 0.645 | 0.327 | 0.625 | 0.567 | 0.577 | 0.589 |
| | | Retentate | −0.542 | −0.280 | 0.396 | −0.252 | 0.350 | −0.038 | 0.124 | −0.094 |
| | | Filtrate | 0.211 | 0.256 | 0.832 | 0.777 | 0.713 | 0.645 | 0.669 | 0.627 |
| | | Corrected cells | 0.151 | 0.271 | 0.960 | 0.309 | 0.925 | 0.788 | 0.951 | 0.473 |
| 368/526 | Riboflavine | Untreated | 0.661 | 0.681 | 0.821 | 0.562 | 0.833 | 0.866 | 0.605 | 0.609 |
| | | Retentate | −0.274 | −0.079 | 0.457 | −0.101 | 0.558 | 0.235 | 0.384 | 0.165 |
| | | Filtrate | 0.590 | 0.623 | 0.957 | 0.671 | 0.875 | 0.896 | 0.915 | 0.729 |
| | | Corrected cells | 0.461 | 0.500 | 0.965 | 0.470 | 0.940 | 0.838 | 0.959 | 0.645 |
| 368/532 | FAD | Untreated | 0.652 | 0.668 | 0.824 | 0.566 | 0.819 | 0.852 | 0.597 | 0.612 |
| | | Retentate | −0.279 | −0.091 | 0.524 | −0.074 | 0.547 | 0.223 | 0.311 | 0.157 |
| | | Filtrate | 0.589 | 0.626 | 0.962 | 0.672 | 0.878 | 0.898 | 0.900 | 0.736 |
| | | Corrected cells | 0.499 | 0.522 | 0.973 | 0.507 | 0.937 | 0.836 | 0.956 | 0.646 |
| 431/535 | FAD | Untreated | 0.843 | 0.802 | 0.861 | 0.480 | 0.826 | 0.763 | 0.776 | 0.548 |
| | | Retentate | 0.483 | 0.295 | 0.007 | 0.496 | 0.405 | 0.615 | −0.145 | 0.420 |
| | | Filtrate | 0.804 | 0.818 | 0.952 | 0.578 | 0.878 | 0.889 | 0.968 | 0.634 |
| | | Corrected cells | 0.920 | 0.624 | 0.927 | 0.703 | 0.957 | 0.942 | 0.982 | 0.584 |
| 452/532 | Riboflavine | Untreated | 0.839 | 0.812 | 0.768 | 0.467 | 0.801 | 0.745 | 0.701 | 0.539 |
| | FAD | Retentate | −0.078 | 0.009 | −0.326 | 0.299 | −0.035 | 0.196 | −0.379 | 0.381 |
| | | Filtrate | 0.820 | 0.832 | 0.949 | 0.571 | 0.885 | 0.889 | 0.961 | 0.637 |
| | | Corrected cells | 0.907 | 0.756 | 0.793 | 0.627 | 0.910 | 0.918 | 0.917 | 0.578 |

TABLE 6
Standard deviation and coefficient of variance for various endogenous biological markers.

| | | | Averages and Standard deviations | | | | | |
|---|---|---|---|---|---|---|---|---|
| | | | Growth | | | Entire culture | | |
| Biomass | | | average | s.d. | c.v. | average | s.d. | c.v. |
| 230/352 | Tryptamine | Untreated | 0.802 | 0.099 | 12% | 0.742 | 0.125 | 17% |
| | | Retentate | 0.782 | 0.115 | 15% | 0.719 | 0.200 | 28% |
| | | Filtrate | 0.894 | 0.058 | 6% | 0.733 | 0.159 | 22% |
| | | Corrected cells | 0.931 | 0.039 | 4% | 0.842 | 0.157 | 19% |
| 263/334 | Phenlylalanie | Untreated | 0.278 | 0.283 | 102% | 0.125 | 0.203 | 162% |
| | Histidine | Retentate | 0.517 | 0.397 | 77% | 0.391 | 0.316 | 81% |
| | | Filtrate | 0.445 | 0.161 | 36% | 0.259 | 0.158 | 61% |
| | | Corrected cells | 0.897 | 0.111 | 12% | 0.809 | 0.188 | 23% |
| 263/397 | Pyridoxine | Untreated | 0.333 | 0.387 | 116% | 0.207 | 0.274 | 132% |
| | | Retentate | 0.006 | 0.258 | 4037% | −0.045 | 0.249 | −557% |
| | | Filtrate | 0.181 | 0.357 | 197% | −0.009 | 0.147 | −1640% |
| | | Corrected cells | 0.856 | 0.189 | 22% | 0.772 | 0.275 | 36% |
| 275/349 | Tryptamine | Untreated | 0.300 | 0.248 | 83% | 0.143 | 0.172 | 120% |
| | | Retentate | 0.517 | 0.291 | 56% | 0.422 | 0.258 | 61% |
| | | Filtrate | 0.516 | 0.164 | 32% | 0.400 | 0.063 | 16% |
| | | Corrected cells | 0.899 | 0.100 | 11% | 0.805 | 0.188 | 23% |
| 275/358 | Trytptophane | Untreated | 0.357 | 0.237 | 66% | 0.217 | 0.178 | 82% |
| | | Retentate | 0.525 | 0.321 | 61% | 0.415 | 0.301 | 72% |
| | | Filtrate | 0.549 | 0.125 | 23% | 0.459 | 0.028 | 6% |
| | | Corrected cells | 0.900 | 0.108 | 12% | 0.792 | 0.227 | 29% |
| 275/448 | NADH | Untreated | 0.600 | 0.301 | 50% | 0.471 | 0.145 | 31% |
| | | Retentate | 0.180 | 0.350 | 194% | −0.016 | 0.225 | −1387% |
| | | Filtrate | 0.464 | 0.378 | 82% | 0.234 | 0.122 | 52% |
| | | Corrected cells | 0.808 | 0.259 | 32% | 0.688 | 0.169 | 25% |
| 275/530 | Riboflavin | Untreated | 0.619 | 0.239 | 39% | 0.555 | 0.144 | 26% |
| | FAD | Retentate | −0.031 | 0.273 | −875% | −0.060 | 0.196 | −327% |
| | | Filtrate | 0.751 | 0.243 | 32% | 0.645 | 0.133 | 21% |
| | | Corrected cells | 0.845 | 0.205 | 24% | 0.757 | 0.166 | 22% |
| 300/400 | ATP | Untreated | 0.401 | 0.378 | 94% | 0.273 | 0.182 | 67% |
| | | Retentate | 0.094 | 0.395 | 421% | −0.059 | 0.285 | −485% |
| | | Filtrate | 0.197 | 0.388 | 197% | 0.001 | 0.116 | 17747% |
| | | Corrected cells | 0.809 | 0.293 | 36% | 0.712 | 0.223 | 31% |
| 323/394 | Pyridoxine | Untreated | −0.302 | 0.291 | −96% | −0.340 | 0.162 | −48% |
| | | Retentate | −0.210 | 0.261 | −125% | −0.302 | 0.173 | −57% |
| | | Filtrate | −0.297 | 0.348 | −117% | −0.456 | 0.133 | −29% |
| | | Corrected cells | 0.735 | 0.379 | 52% | 0.571 | 0.222 | 39% |
| 350/445 | NADH | Untreated | 0.509 | 0.238 | 47% | 0.406 | 0.142 | 35% |
| | | Retentate | 0.019 | 0.398 | 2060% | −0.183 | 0.101 | −55% |
| | | Filtrate | 0.442 | 0.316 | 71% | 0.323 | 0.167 | 52% |
| | | Corrected cells | 0.733 | 0.406 | 55% | 0.469 | 0.237 | 51% |
| 356/598 | Sanguinarine | Untreated | −0.445 | 0.121 | −27% | −0.183 | 0.230 | −126% |
| | | Retentate | −0.073 | 0.261 | −357% | −0.189 | 0.157 | −83% |
| | | Filtrate | 0.723 | 0.143 | 20% | 0.685 | 0.119 | 17% |
| | | Corrected cells | 0.842 | 0.192 | 23% | 0.585 | 0.228 | 39% |
| 360/465 | Autofluorescence | Untreated | 0.536 | 0.161 | 30% | 0.470 | 0.129 | 27% |
| | | Retentate | 0.082 | 0.433 | 528% | −0.166 | 0.118 | −71% |
| | | Filtrate | 0.606 | 0.272 | 45% | 0.576 | 0.224 | 39% |
| | | Corrected cells | 0.747 | 0.397 | 53% | 0.460 | 0.235 | 1% |
| 368/526 | Riboflavin | Untreated | 0.730 | 0.115 | 16% | 0.680 | 0.134 | 20% |
| | | Retentate | 0.281 | 0.377 | 134% | 0.055 | 0.170 | 311% |
| | | Filtrate | 0.834 | 0.166 | 20% | 0.730 | 0.119 | 16% |
| | | Corrected cells | 0.831 | 0.247 | 30% | 0.613 | 0.168 | 27% |
| 368/532 | FAD | Untreated | 0.723 | 0.116 | 16% | 0.675 | 0.125 | 19% |
| | | Retentate | 0.276 | 0.385 | 140% | 0.054 | 0.160 | 298% |
| | | Filtrate | 0.832 | 0.166 | 20% | 0.733 | 0.119 | 16% |
| | | Corrected cells | 0.842 | 0.229 | 27% | 0.627 | 0.152 | 24% |
| 431/535 | FAD | Untreated | 0.827 | 0.036 | 4% | 0.648 | 0.158 | 24% |
| | | Retentate | 0.187 | 0.304 | 163% | 0.457 | 0.134 | 29% |
| | | Filtrate | 0.901 | 0.075 | 8% | 0.730 | 0.147 | 20% |
| | | Corrected cells | 0.946 | 0.029 | 3% | 0.713 | 0.160 | 22% |
| 452/532 | Riboflavin | Untreated | 0.777 | 0.059 | 8% | 0.641 | 0.164 | 26% |
| | FAD | Retentate | −0.204 | 0.173 | −85% | 0.221 | 0.161 | 73% |
| | | Filtrate | 0.904 | 0.065 | 7% | 0.732 | 0.152 | 21% |
| | | Corrected cells | 0.882 | 0.060 | 7% | 0.720 | 0.152 | 21% |

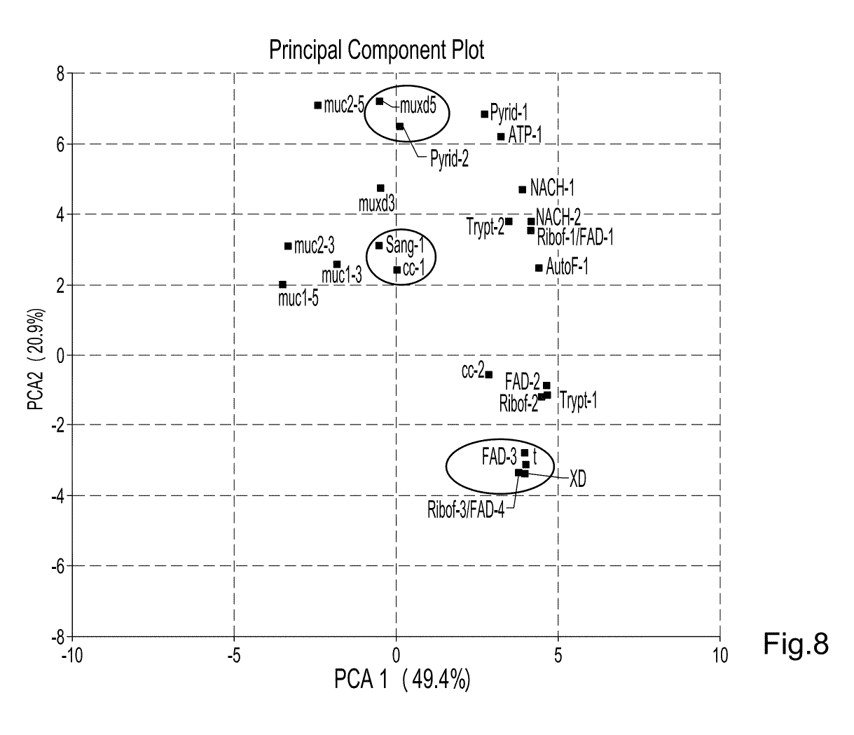

Identification of biological markers with multi-variable analysis. A Principal Component Analysis was performed on the entire data set comprising the culture variables (such as biomass concentration, cell concentration, biomass accumulation rate, cell proliferation, etc.) and the potential biological markers. Correlations between culture variables and fluorescence couples were identified by the proximity of the variables on the graphic of PC2 vs PC1 as shown on FIG. 8.

### EXAMPLE IV

### Prediction of Cell Culture Parameters Using the Biological Markers

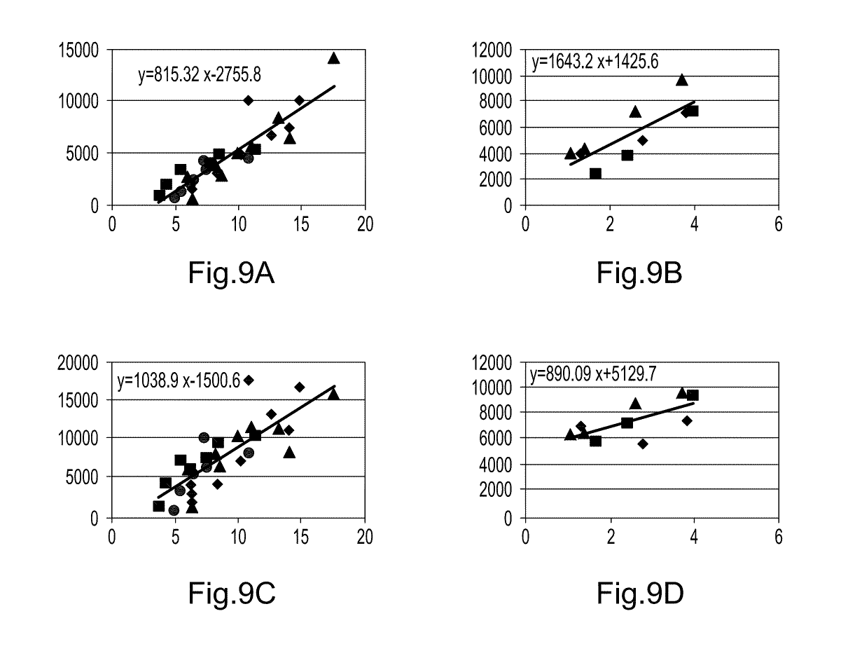

By plotting the measurement of fluorescence obtained for the markers identified in Example II in function of the offline parameter, it is possible to obtain a mathematical relationship between the marker and the parameter. The results obtained with tryptamine and riboflavin are shown in FIG. 9. As indicated on this figure, the relationship between the fluorescent signal of the marker (y) and the parameter (x) are as following:
Tryptamine (y) as a marker for biomass concentration (x) in the filtrate

y=815.35x−2755.8
Tryptamine (y) as a marker for biomass concentration (x) in the raw sample

y=1038.9x−1500.6
Riboflavin (y) as a marker for the rate of cellular proliferation (x) in the filtrate

y=1643.2x+1425.6
Riboflavin (y) as a marker for the rate of cellular proliferation (x) in the raw sample

y=890.09x+5129.7

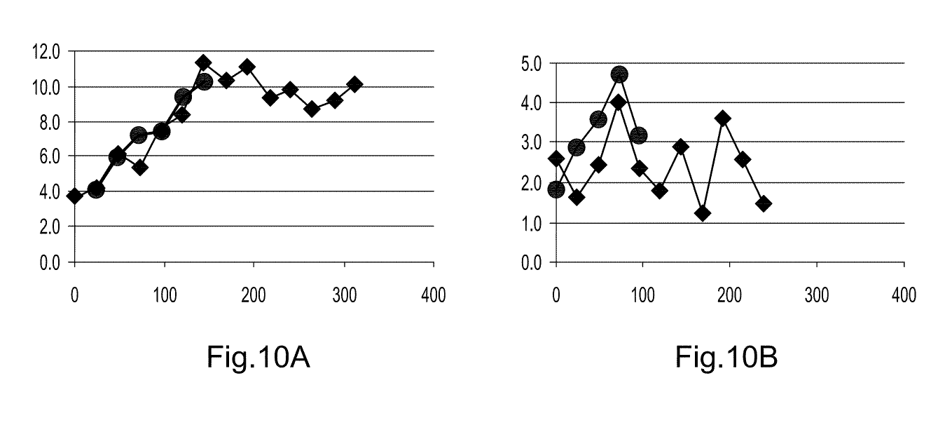

Consequently, the slope between the measure of fluorescence for the marker and the cell culture parameter has been determined. For the biomass concentration, the slope was evaluated at 0.001 (g/L)/RFU. For the rate of cellular proliferation, the slope was evaluated at 0.0005 (10⁹ cells/L)/RFU. These slopes enabled the prediction of a cell culture parameter based on a fluorescence reading. Results of this prediction are shown in FIG. 10.

### EXAMPLE V

### Results Obtained with other Cell Suspensions

The methodology described in Example I to IV has been applied with other cell suspensions, such as Arabidopsis thaliana (another plant species), Streptomyces scabies (a filamentous bacteria) and RAJI cells (B lymphocytes) that have been cultured in vitro. The results indicated that specific biomarkers can be obtained for these cell cultures. The results also showed that this methodology could be applied to other plant cell culture, prokaryotic cell cultures and other eukaryotic (animal/mammal) cell culture.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method of identifying an endogenous fluorescent marker associated with a parameter of a cell culture, said method comprising:
obtaining an optical imprint of a chemically purified form of the endogenous fluorescent marker by: (i) irradiating the endogenous fluorescent marker with a light source emitting at least at one specific excitation wavelength to generate a fluorescent signal from the endogenous fluorescent biological marker, and (ii) quantifying, at least at one specific emission wavelength, the intensity of the fluorescent signal from (i);
obtaining a plurality of fluorescent signals from the cell culture as a function of time, wherein the plurality of fluorescent signals are obtained by: (iii) irradiating a cell culture or derivative thereof with a light source emitting at least at one specific excitation wavelength to generate a fluorescent signal from the cell culture or the derivative thereof, and (iv) quantifying, at least at one specific emission wavelength, the intensity of the fluorescent signal from the cell culture or the derivative thereof;
obtaining a plurality of values for the parameter of the cell culture as a function of time;
determining which of the plurality of fluorescent signals of the cell culture are associated with the optical imprint of the chemically purified form of the endogenous fluorescent marker to provide a first set of fluorescent signals;
determining which of the plurality of fluorescent signals of the cell culture are associated with the plurality of values of the parameter of the cell culture to provide a second set of fluorescent signals; and
characterizing the endogenous fluorescent marker as being indicative of the parameter of the cell culture when the first set of fluorescent signals is correlated to the second set of fluorescent signals.

2. The method of claim 1, wherein the optical imprint of the endogenous biological marker comprises at least one fluorescent peak obtained from a three-dimensional fluorescent spectra by (a) irradiating the endogenous fluorescent marker with a light source emitting at a plurality of specific excitation wavelengths to generate a plurality of fluorescent signals from the endogenous fluorescent biological marker; (b) quantifying, from a corresponding plurality of specific emission wavelengths, the intensity of the fluorescent signals from (a); and (c) identifying at least one of the plurality of fluorescent signals obtained in step (b) as the fluorescent peak.

3. The method of claim 1, wherein the plurality of fluorescent signals from the cell culture are obtained online.

4. The method of claim 1, wherein the plurality of fluorescent signals from the cell culture are obtained with a probe.

5. The method of claim 1, wherein said cell culture is an untreated sample of the cell culture, and said derivative thereof is at least one of a filtered sample of the cell culture and a resuspended retentate of the filtered sample.

6. The method of claim 1, wherein the correlation is made by comparing graphical representations of the first set of fluorescent signals and the second set of fluorescent signals and by visually identifying graphical similarities between the first set of fluorescent signals and the second set of fluorescent signals.

7. The method of claim 1, wherein the correlation is made by a numerical technique, when a modulation in the first set of fluorescent signals, as a function of time, is associated to a corresponding modulation in the second set of fluorescent signals, as a function of time.

8. The method of claim 15, wherein the numerical technique is a covariance coefficient technique.

9. The method of claim 1, wherein the correlation is made by a multivariable technique, when a modulation in the first set of fluorescent signals, as a function of time, is associated to a corresponding modulation in the second set of fluorescent signals, as a function of time.

10. The method of claim 1, wherein the parameter is at least one of biomass concentration, rate of biomass accumulation, cellular concentration, rate of cellular proliferation, accumulation of a by-product and consumption of a nutrient.
